(19) United States
(12) Patent Application Publication
(10) Pub. No.: US 2024/0082272 A1
(43) Pub. Date: Mar. 14, 2024

(54) COMPOSITIONS AND METHODS FOR TREATING WNT-DRIVEN CANCER

(71) Applicant: The Regents of the University of Michigan, Ann Arbor, MI (US)

(72) Inventors: ANTONIO LERARIO, Ann Arbor, MI (US); GARY HAMMER, Ann Arbor, MI (US); DIPIKA MOHAN, Ann Arbor, MI (US)

(21) Appl. No.: 18/038,778

(22) PCT Filed: Dec. 9, 2021
(86) PCT No.: PCT/US2021/062519

§ 371 Date: May 17, 2023

Related U.S. Application Data
(60) Provisional application No. 63/123,264, filed on Dec. 9, 2020.

(51) Int. Cl. A61K 31/675; A61K 31/4155; A61K 31/423; A61K 31/437; A61K 31/4439; A61K 31/444; A61K 31/4725; A61K 31/496; A61K 31/519; A61K 31/5377; A61P 35/00

(52) U.S. Cl. CPC A61K 31/675; A61K 31/4155; A61K 31/423; A61K 31/437; A61K 31/4439; A61K 31/444; A61K 31/4725; A61K 31/496; A61K 31/519; A61K 31/5377; A61P 35/00

(57) ABSTRACT

The present disclosure relates to compositions, systems, and methods for treating cancer. In particular, the present disclosure relates to compositions, systems, and methods for targeting oncogenic, Wnt-dependent transcriptional programs in cancers, utilizing as an example adrenocortical carcinoma stratification to treat adrenocortical carcinoma and drugs which have utility for patients stratified by these means.

### FIELD OF THE DISCLOSURE

The present disclosure relates to compositions, systems, and methods for treating cancer. In particular, the present disclosure relates to compositions, systems, and methods for targeting oncogenic, Wnt-dependent transcriptional programs in cancers, utilizing as an example adrenocortical carcinoma stratification to treat adrenocortical carcinoma and drugs which have utility for patients stratified by these means.

### BACKGROUND OF THE DISCLOSURE

The Wnt/β-catenin pathway (also known as the canonical Wnt pathway), is rendered constitutively active through somatic alterations in ˜20% of all human cancers. Efforts to therapeutically inhibit this pathway have been largely unsuccessful. Wnt/β-catenin signaling is required for stem/progenitor cell maintenance in most tissues, and targeting this pathway clinically almost invariably results in on-target intolerable systemic toxicities in organs that undergo frequent, Wnt-dependent renewal. Clinical investigation of Wnt-pathway-targeting agents that hit degenerate components of this pathway expressed in all cells with activation of this program (e.g. tankyrase inhibitors) have been discontinued due failure in Phase I and II clinical trials.

Limiting systemic toxicity associated with disruption of Wnt/β-catenin signaling requires harnessing context-specific actions of this pathway. An example of a cancer suitable for targeting this pathway is adrenocortical carcinoma (ACC).

ACC is a rare malignancy with an overall dismal prognosis. Treatment options for ACC are limited, and surgery is the only therapy that can provide long-term remission and cure. Despite surgery, many patients with early-stage disease develop metastases post-operatively and therefore require systemic treatment. For this reason, following margin-free surgical resection, adjuvant therapy with the adrenolytic compound mitotane is now part of the standard care for most ACC patients; however, current pharmacologic treatment options are highly limited and leave a major unmet medical need for additional options. Recent studies confirm that mitotane is only marginally effective while highly toxic. Therapeutic serum levels of mitotane typically take several months of drug administration to achieve, and up to 90% of patients will inevitably recur during adjuvant mitotane therapy after surgery or progress during mitotane therapy for non-resectable disease (either during or following this dosage escalation window). Furthermore, the efficacy of cytotoxic chemotherapy for nonresectable disease is similarly limited and side effects are significant. As a result, there is a critical unmet medical need for new systemic therapies which are safer, more effective, or both, than current options for patients. Application of these therapies necessarily relies on appropriate molecular stratification of patient samples, which is needed.

### SUMMARY OF THE DISCLOSURE

The present disclosure relates to compositions, systems, and methods for treating cancer. In particular, the present disclosure relates to compositions, systems, and methods for targeting oncogenic, Wnt-dependent transcriptional programs in cancers, utilizing as an example adrenocortical carcinoma stratification to treat adrenocortical carcinoma and drugs which have utility for patients stratified by these means.

The present disclosure provides improved treatment methods for subjects with cancer that exhibit constitutive activation of the Wnt/β-catenin pathway such as, including but not limited to, COC2 or COC3/CIMP-high ACC. By provided target therapies, alone or in combination, the present disclosure allows therapies to be administered at lower doses to limit undesirable and potentially unsafe systemic effects.

For example, in some embodiments, provided herein is a method for treating adrenocortical carcinoma (ACC), comprising: administering at least one agent that blocks activation of the Wnt/β-catenin pathway to a subject identified as having COC2 or COC3/CIMP-high ACC.

Further embodiments provide a method for treating cancer, comprising: administering at least one agent that blocks activation of the Wnt/β-catenin pathway to a subject identified as having a cancer that exhibits constitutive activation of the Wnt/β-catenin pathway.

Certain embodiments provide a method for treating cancer, comprising: administering an agent that blocks activation of the Wnt/β-catenin pathway to a subject identified as having constitutive activation of the Wnt/β-catenin pathway in a sample isolated from the subject.

Yet other embodiments provide a method for treating cancer in a subject, comprising: a) identifying the subject constitutive activation of the Wnt/β-catenin pathway by obtaining or having obtained a sample from the subject; and measuring the level of activation of the Wnt/β-catenin pathway in the sample; and b) administering an agent that blocks activation of the Wnt/β-catenin pathway to the subject when the sample exhibits constitutive activation of the Wnt/β-catenin pathway.

Still further embodiments provide a method for treating cancer in a subject, comprising a) determining the level of activation of the Wnt/β-catenin pathway in a sample from the subject; b) identifying subjects with constitutive activation of the Wnt/β-catenin pathway in the sample; and c) administering an agent that blocks activation of the Wnt/β-catenin pathway to the subject identified as having constitutive activation of the Wnt/β-catenin pathway.

Additional embodiments provide the use of an agent that blocks activation of the Wnt/β-catenin pathway to treat ACC in a subject identified as having COC2 or COC3/CIMP-high ACC.

Also provided is an agent that blocks activation of the Wnt/β-catenin pathway for use in treating ACC in a subject identified as having a COC2 or COC3/CIMP-high ACC. In some embodiments, the ACC exhibits constitutive activation of the Wnt/β-catenin pathway. In some embodiments, the cancer is COC2 or COC3/CIMP high ACC. In some embodiments, the sample comprises cancer cells or tissue.

The present disclosure is not limited to particular agents. In some embodiments, the agent disrupts binding of SF1/β-catenin complexes to DNA; disrupts transcriptional activation of SF1/β-catenin, and/or disrupts binding of SF1 to β-catenin. Examples of suitable agents include but are not limited to, an EZH2 inhibitor, a CBP inhibitor, an SF1 inhibitor, or combinations thereof. Examples of agents include but are not limited to, an antibody, a nucleic acid, and a small molecule. In some embodiments, the EZH2 inhibitor is EED226, EPZ-6438, 3-Deazaneplanocin, DZNep, EPZ005687, GSK503, GSK343, GSK126, EL1, or CPI-169. In some embodiments, the CBP inhibitor is ICG-001, PRI-724, A485, C646,

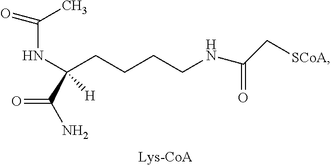

or garcinol. In some embodiments, the SF1 inhibitor is SID7969543 or SID7970631.

Additional embodiments will be apparent to persons skilled in the relevant art based on the teachings contained herein.

### DESCRIPTION OF THE DRAWINGS

FIG. 1. Complete EZH2 interactome from NCI-H295R nuclear lysates reveals context- and tissue-specific binding partners. Complete proteome from MS of EZH2-directed nuclear co-IP reveals that EZH2 interactome is comprised predominantly of classical PRC2 and PCL proteins, complexes implicated in DNA repair, and novel binding partners critical for adrenocortical differentiation and cancer (notably: NR4A3, NR4A1, and p.S45P β-catenin). y-axis depicts spectral counts (SpC). EZH2 binding to p. S45P β-catenin was validated by targeted nuclear co-IP (n=5, data not shown).

FIG. 2. EZH2/β-catenin binding is PRC2 independent and off chromatin. Left, EZH2-directed nuclear co-IP consistently retrieves SUZ12 and β-catenin, while SUZ12-directed nuclear co-IP only retrieves EZH2. These data indicate EZH2/β-catenin interact in an off chromatin complex. Representative western blot shown. On top row, Input=10% input, No Ab=IP performed with no antibody, IgG=IP performed with negative control IgG, EZH2=IP performed with antibody against EZH2, SUZ12=IP performed with antibody against SUZ12. Right, consistent with our nuclear co-IP results, overlap of NCI-H295R baseline H3K27me3, EZH2, β-catenin peaks reveals β-catenin only minimally interacts with PRC2 and EZH2 on chromatin.

FIG. 3. EZH2/β-catenin complex is present in the nucleus of adrenocortical zG/upper zF cells. Proximity ligation assay (PLA) was performed on murine adrenals to examine EZH2/β-catenin binding in vivo. PLA was performed with no antibodies (negative control, left), two antibodies against β-catenin (positive control, middle), or antibodies recognizing EZH2 and β-catenin (right). The images in this figure are from a ˜7 week old female that has undergone ACTH-dependent adrenal regeneration and experienced complete cortical renewal. Bar=100 uM. PLA signal (reddish/pink dots, sub-nuclear in size) for EZH2/β-catenin is present, nuclear, and stronger in the zG/upper zF, mirroring the Wnt/β-catenin gradient (middle). No antibody is a standard negative control for PLA; additional studies not shown here show little to no signal when slides are incubated with antibodies only from a single species. Sections are shown with capsule aligned to the top of the field.

FIG. 4. EZH2/SUZ12 and EZH2/β-catenin are preserved even after EZH2i. EZH2-directed nuclear complex co-IP in vehicle- (left) or EZH2i-treated (right, boxed for each drug) cells demonstrates the persistence of EZH2/SUZ12 and EZH2/β-catenin complexes even after EZH2i. Representative experiment shown (n=2 biological replicates). On top row, In=10% input, IgG=IP with negative control Rb IgG, EZH2=IP with EZH2. Band in EPZ-6438 IgG EZH2/SUZ12 blots is a non-specific band that occasionally emerges at this weight when using rabbit antibodies for IP and western detection and does not represent bona fide EZH2/SUZ12 signal.

FIG. 5. CIMP-high ACC possess hyperactivation of zF differentiation, Wnt/β-catenin-dependent programming, and proliferation. GSVA (Hänzelmann et al., 2013) was used to calculate the expression score of genes that define adrenal differentiation (Zheng et al., 2016) or genes which were identified by Independent Component Analysis (ICA, (Biton et al., 2014)) to be regulated in a cell-cycle- or Wnt-dependent manner across ACC-TCGA (supported by significantly higher component score [p<0.05, Mann Whitney] in tumors with driver alterations in the cell cycle or Wnt pathway). The average score for CIMP-low, CIMP-int, and CIMP-high tumors along each axis is plotted in this radar plot, with values mapped onto an arbitrary scale of 1-5 dictating position along each axis. CIMP-low tumors have uniformly low expression of adrenal differentiation, Wnt programming and cell cycle activation. CIMP-intermediate tumors have relatively higher activation of these programs, and maximize Wnt signaling. CIMP-high tumors have the highest activation of all three programs.

FIG. 6. Forskolin induces faithful zF differentiation and steroidogenesis in NCI-H295R. Left, diffTF analysis integrating RNA-seq and ATAC-seq data from NCI-H295R treated with forskolin for 48 hours vs. vehicle control reveals that forskolin induces a prominent induction of immediate early response programs (Fos/Jun) at the expense of shutting down endogenous Wnt/β-catenin signaling (TCF). The induction of immediate early response programs is consistent with known mechanisms of action of PKA. Right, also consistent with known roles of PKA signaling, forskolin potently induced expression of steroidogenic enzymes, consistent with induction of faithful zF differentiation in NCI-H295R.

FIG. 7. EZH2i reverses zF differentiation. A. RNA-seq from NCI-H295R treated with EZH2i demonstrates that EZH2i represses expression of steroidogenic enzymes induced by forskolin (FIG. 7) and indicative of steroidogenic differentiation. B. Two classes of EZH2i repress HSD3B2 in a dose-dependent manner, prior to the IC-50 (indicated by the red arrow; in the case of EPZ-6438 IC-50 is at 62 uM which was not tested in this experiment). Representative experiment shown, n>3. C. NCI-H295R were pretreated with EZH2i for 96 hours at the indicated doses (either the IC-50 or half of the IC-50). After EZH2i, media was changed for media containing 10 uM forskolin. After 48 hours of forskolin stimulation, cells were harvested for analysis of gene expression by qPCR. For both EZH2i, EZH2i pre-treatment disrupted forskolin's induction of steroidogenic enzymes (HSD3B2) and repression of canonical Wnt targets (APCDD1, LGR5). n>2.

FIG. 8. EZH2i reverses the core transcriptional features of CIMP-high ACC. Wnt, cell cycle, and adrenal differentiation scores in NCI-H295R were quantified from RNA-seq derived from baseline (Vehicle) NCI-H295R or after EZH2i or forskolin (FSK) administration using GSVA as in FIG. 6, and demonstrates that EZH2i reverses all three CIMP-high defining programs, while forskolin increases adrenal differentiation at the expense of Wnt signaling and cellular proliferation.

FIG. 9. β-catenin binds SF1 and TCF/LEF motifs at active and accessible chromatin genomewide. Measurement of genome-wide distribution of β-catenin, H3K27ac, and chromatin accessibility at baseline β-catenin binding sites in NCI-H295R reveals that the vast majority of β-catenin peaks are decorated with H3K27ac and are also accessible. Strikingly, motif enrichment for β-catenin identified a highly significant enrichment for regions bearing the SF1 motif (depicted in the bar graph, left, as NR5A2), far exceeding the enrichment for regions bearing canonical LEF motifs. This data suggested β-catenin may co-regulate SF1-dependent the transcriptional landscape.

FIG. 10. SF1-directed IP-MS identifies β-catenin as the dominant binding partner. Complete proteome from MS of SF1-directed nuclear co-IP identifies β-catenin as a dominant binding partner. y-axis depicts spectral counts (SpC). SF1 binding to β-catenin was validated by targeted nuclear co-IP (n=2). SF1 antibody used for IP-MS is a custom polyclonal antibody purified from rabbit sera. Iron binding and complement/coagulation components (grey) likely reflect contaminants present in the antibody solvent.

FIG. 11. SF1/β-catenin is zonally distributed in the murine adrenal cortex. PLA was performed on murine adrenals to examine SF1/β-catenin binding in vivo. PLA was performed with no antibodies, two antibodies against β-catenin, antibodies recognizing EZH2 and β-catenin (FIG. 4), or antibodies recognizing SF1 and β-catenin (here). The images in this figure are from the same ˜7 week old female that has undergone ACTH-dependent adrenal regeneration and experienced complete cortical renewal in FIG. 4. Bar=100 uM. PLA signal (reddish/pink dots, sub-nuclear in size) for SF1/β-catenin is abundant, nuclear, and stronger in the zG/upper zF, mirroring the Wnt/β-catenin gradient (FIG. 4). Sections are shown with capsule aligned to the top of the field.

FIG. 12. SF1/β-catenin overlap genome-wide. SF1 occupies many sites in the NCI-H295R genome, with >20,000 peaks. β-catenin's binding profile is more restricted, and nearly half of all β-catenin peaks colocalize with SF1.

FIG. 13. SF1/β-catenin predominantly occupy distal CREs. Characteristics of SF1/β-catenin binding sites identifies that 65% of peaks are >1000 bp away from a TSS and are therefore distal.

FIG. 14. SF1/β-catenin coordinate lineage-defining super-enhancers in NCI-H295R. Comparison of NCI-H295R SE (identified using ROSE) and physiological adrenal SE (obtained from 3DIV analysis on ENCODE samples) identifies many novel SE in NCI-H295R. Despite the small overlap between adrenal SE and NCI-H295R SE, 93% of adrenal SE still retain H3K27ac in NCI-H295R, indicating that adrenal SE are downgraded in NCI-H295R but not totally decommissioned. Evaluation of SF1/β-catenin occupancy at NCI-H295R reveals that 72% of NCI-H295R SE are regulated by both SF1 and β-catenin. This is in striking contrast to adrenal SE, in which only 32% possess SF1 and β-catenin in NCI-H295R. These data indicate that SF1/β-catenin in NCI-H295R are monopolizing machinery required for SE establishment (for example CBP) at the expense of other transcriptional programs.

FIG. 15. SF1/β-catenin co-targets are more accessible in CIMP-high ACC. Chromatin accessibility signal at SF1/β-catenin co-targets was measured in ACC-TCGA samples with ATAC-seq data (n=9), sorted by CIMP status. Though only few ACC-TCGA samples have ATAC-seq data, this analysis revealed that SF1/β-catenin co-targets are increasingly accessible in CIMP-intermediate and CIMP-high ACC.

FIG. 16. EZH2i represses expression of genes putatively regulated by SF1/β-catenin enhancers. Genes putatively targeted by active SF1/β-catenin enhancers were identified by overlapping human adrenal promoter capture Hi-C contact tables (Jung et al., 2019) with H3K27ac and then overlapping enhancers with the consensus SF1/β-catenin peak set. More than a third of all genes downregulated with EZH2i are putatively regulated by SF1/β-catenin enhancers.

FIG. 17. EZH2i evicts SF1 and β-catenin genome wide. Heatmap depicts SF1, β-catenin, EZH2, H3K27ac, and chromatin accessibility (ATAC-seq) signal at baseline SF1 peaks at baseline (−) or with EZH2i (+). EZH2i in NCI-H295R leads to global eviction of SF1 and β-catenin at baseline SF1 binding sites coincident with aberrant recruitment of EZH2 and decreased chromatin accessibility. Not shown, 56% of baseline β-catenin peaks do not possess SF1, and EZH2i also evicts β-catenin from those sites.

FIG. 18. SF1/β-catenin recruitment to HSD3B2 and NR5A1 super-enhancers is disrupted by EZH2i and associated with a decrease in gene expression. Genome browser view of H3K27ac, β-catenin, and SF1 signal at baseline (−) or with EZH2i (+) at SE spanning the NR5A1 (top) or HSD3B2 (bottom) loci demonstrates diminishing SF1 signal and disappearing β-catenin peaks, associated with a substantial and significant decrease in gene expression (right, from NCIH295R RNA-seq). SE were identified using ROSE and assigned to NR5A1 and HSD3B2 by overlapping human adrenal promoter capture Hi-C contact tables (Jung et al., 2019).

FIG. 19. EZH2i disrupts global super-enhancer programming. SE identification before and after EZH2i demonstrates that EZH2i erased nearly 50% of SE, and retained SE lost SF1/β-catenin coordinate control After EZH2i, only 35% of retained SE are bound by both SF1 and β-catenin.

FIG. 20. EZH2i and CBPi are synergistic in ACC cell lines. NCI-H295R, ATC7L, and Y1 were treated with increasing doses of CBPi (PRI-724), alone or in combination with EZH2i at the IC-50 dose for each cell line (EZH2i viability curves for ATC7L and Y1 not shown). EZH2i and CBPi induce synergistic (S) loss of viability at at increasing doses of CBPi in all ACC cell lines, indicating that EZH2 and CBP may redundantly coordinate epigenetic programming.

FIG. 21. EZH2i and CBPi redundantly disrupt the NCI-H295R transcriptome. A. Left, Venn diagram depicting genes that are differentially expressed following 96-hour IC-50 administration of EZH2i (EPZ-6438) or CBPi (PRI-724) in NCI-H295R reveals highly significant overlap, with ˜70% of differentially expressed genes in each set overlapping. Right, gene expression changes induced by EZH2i and CBPi are strongly correlated. B. Like EZH2i, CBPi induces potent downregulation of steroidogenesis, consistent with induction of a dedifferentiation program. C. Representative experiment measuring HSD3B2 expression by qPCR after CBPi administration reveals CBPi induces dose-dependent downregulation of steroidogenic enzymes like HSD3B2, even at doses under the IC-50 (indicated by the red arrow).

FIG. 22. CBPi, like EZH2i, reverses CIMP-high-defining transcriptional programs. Adrenal differentiation, Wnt, and cell cycle scores for CBPi, EZH2i, and Fsk calculated by GSVA (Hänzelmann et al., 2013) and graphed as in FIGS. 6 and 9.

FIG. 23. Strategies for disruption of tissue-specific oncogenic programs. β-catenin-dependent transcriptional programming with tissue-specific transcription factors (TF) may be rendered constitutively active through a variety of mechanisms in cancer. β-catenin/tissue-specific factors may regulate transcription through binding at active enhancers and/or promoters. Disrupting these programs by disrupting coactivator/tissue-specific TF binding to the genome (A), disrupting coactivator/tissue-specific TF-dependent transcriptional activation (B), or disrupting coactivator binding to tissue-specific TFs (C) may be therapeutically efficacious.

FIG. 24. Tissue-specific β-catenin complexes across human adrenocortical tumors. PLA was performed on a tissue microarray of benign adrenal adenomas (ACA), primary adrenal cancer (ACC), and metastatic adrenal cancer (metastases). PLA signal was quantified per nucleus using a custom macro in ImageJ. EZH2/β-catenin (EB) and SF1/β-catenin (SB) complexes persist through benign and malignant tumorigenesis, and an increased abundance of EB relative to SB in malignancy was present.

FIG. 25. Tissue-specific β-catenin complexes persist in mouse models of adrenal cancer. PLA signal from a representative lung metastasis derived from a genetically engineered mouse model of adrenal cancer (bearing an activating alteration in Ctnnb1, encoding β-catenin, and inactivating alteration in Trp53, encoding negative cell cycle regulator p53) reveals expression of nuclear SF1/β-catenin and EZH2/β-catenin complexes in bona fide metastatic tissue (identified by histology of metastases compared to lung epithelium and by expression of SF1 by PLA in positive control panels at the top of the figure). Bar represents 100 microns.

FIG. 26. EZH2 inhibition in vivo disrupts tumor growth and differentiation through erasure of tissue-specific β-catenin complexes. EZH2 inhibition (EZH2i=200 mg/kg EPZ-6438 PO daily) in a subcutaneous, immunodeficient xenograft model of adrenal cancer decreases tumor growth (A) and proliferation rate (B, Ki67 index measured using immunohistochemistry with hematoxylin counterstain and quantified per nucleus using a custom macro in ImageJ) compared to vehicle administration. EZH2 inhibition induces dedifferentiation compared to vehicle administration by decreasing expression of SF1 (C, SF1 expression measured using immunohistochemistry with hematoxylin counterstain and quantified per nucleus using a custom macro in ImageJ) (D), PLA signal quantified as in FIG. 24), with retention of off chromatin EZH2/β-catenin (E, PLA signal quantified as in FIG. 24).

### DEFINITIONS

To facilitate an understanding of the present disclosure, a number of terms and phrases are defined below:

As used herein, the term “sensitivity” is defined as a statistical measure of performance of an assay (e.g., method, test), calculated by dividing the number of true positives by the sum of the true positives and the false negatives.

As used herein, the term “specificity” is defined as a statistical measure of performance of an assay (e.g., method, test), calculated by dividing the number of true negatives by the sum of true negatives and false positives.

As used herein, the term “informative” or “informativeness” refers to a quality of a marker or panel of markers, and specifically to the likelihood of finding a marker (or panel of markers) in a positive sample.

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body. As used herein, the term “metastasized ACC cancer cells” is meant to refer to ACC cancer cells which have metastasized.

The term “neoplasm” as used herein refers to any new and abnormal growth of tissue. Thus, a neoplasm can be a non-malignant neoplasm, a premalignant neoplasm or a malignant neoplasm. The term “neoplasm-specific marker” refers to any biological material that can be used to indicate the presence of a neoplasm. Examples of biological materials include, without limitation, nucleic acids, polypeptides, carbohydrates, fatty acids, cellular components (e.g., cell membranes and mitochondria), and whole cells.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, β-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP).

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example, a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As used herein, “methylation” refers to cytosine methylation at positions C5 or N4 of cytosine, the N6 position of adenine, or other types of nucleic acid methylation. In vitro amplified DNA is unmethylated because in vitro DNA amplification methods do not retain the methylation pattern of the amplification template. However, “unmethylated DNA” or “methylated DNA” can also refer to amplified DNA whose original template was unmethylated or methylated, respectively.

“Methylation status” refers to the presence, absence, and/or quantity of methylation at a particular nucleotide or nucleotides within a portion of DNA. The methylation status of a particular DNA sequence (e.g., a gene marker or DNA region as described herein) can indicate the methylation state of every base in the sequence or can indicate the methylation state of a subset of the base pairs (e.g., of one or more cytosines) within the sequence, or can indicate information regarding regional methylation density within the sequence without providing precise information of where in the sequence the methylation occurs. The methylation status can optionally be represented or indicated by a “methylation value.” A methylation value can be generated, for example, by quantifying the amount of intact DNA present following restriction digestion with a methylation dependent restriction enzyme or by comparing amplification profiles after bisulfite reaction or by comparing sequences of bisulfite-treated and untreated DNA. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full-length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences”. Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) processed transcript. The mRNA functions during translation to specify the sequence or order of amino acids in a nascent polypeptide.

The term “locus” as used herein refers to a nucleic acid sequence on a chromosome or on a linkage map and includes the coding sequence as well as 5′ and 3′ sequences involved in regulation of the gene.

### DETAILED DESCRIPTION OF THE DISCLOSURE

The present disclosure relates to compositions, systems, and methods for treating cancer. In particular, the present disclosure relates to compositions, systems, and methods for targeting oncogenic, Wnt-dependent transcriptional programs in cancers, utilizing as an example adrenocortical carcinoma stratification to treat adrenocortical carcinoma and drugs which have utility for patients stratified by these means.

Recent landmark multiplatform molecular profiling studies of human cancers have identified that a core hallmark of this disease is disruption of homeostatic transcriptional and epigenetic programming. This is achieved through recurrent somatic alterations in transcription factors, epigenetic machinery, and transcriptional coactivators. One of such examples is the Wnt/β-catenin pathway (also known as the canonical Wnt pathway), rendered constitutively active through somatic alterations in ˜20% of all human cancers. The high frequency of mutations in the Wnt pathway have made it a promising therapeutic target, however efforts to inhibit this pathway have been largely unsuccessful. Wnt/β-catenin signaling is required for stem/progenitor cell maintenance in most human tissues, and targeting this pathway clinically almost invariably results in on-target systemic toxicities in organs with rapid turnover that require Wnt signaling, e.g. the colon. It is therefore not surprising that clinical investigation of many Wnt-pathway-targeting agents that hit degenerate components of this pathway expressed in all cells with activation of this program (e.g. tankyrase inhibitors) have been discontinued due failure in Phase I and II clinical trials.

Limiting systemic toxicity associated with disruption of Wnt/β-catenin signaling utilizes an approach that harnesses context-specific actions of this pathway. Through comprehensive molecular profiling studies, experiments described herein have identified a novel, tissue-specific axis of the Wnt/β-catenin program that relies on a physical interaction between β-catenin and tissue-specific transcription factors. β-catenin is recruited to active enhancers in a tissue-specific transcription-factor dependent manner independent of degenerate canonical Wnt signaling. Perturbation of this interaction (through multiple pharmacological approaches) causes loss of tissue-specific transcriptional programming, reduces viability, and diminishes sustained proliferation potential in vitro. These data support that the ultimate consequence of Wnt pathway alterations in Wnt-driven cancers is co-optation and mis-engagement tissue-specific transcription factors to reinforce pro-proliferative oncogenic programming.

Accordingly, provided herein are strategies to disrupt tissue-specific engagement of β-catenin that preferentially target the oncogenic roles of this program and spare other tissues, increasing the therapeutic index and likelihood of success targeting this pathway in future clinical trials.

Mutations leading to constitutive activation of the Wnt/β-catenin pathway are recurrent in adrenocortical carcinoma (ACC), occurring in approximately 40% of primary tumors.

Recent comprehensive genomics studies such as The Cancer Genome Atlas study on ACC (ACC-TCGA) indicated that ACC may be better stratified using molecular stratification rather than proliferation measurements (KI67 or mitotic counts). ACC-TCGA demonstrated that ACC is a molecularly heterogeneous disease, comprised largely of 3 distinct molecular subtypes—COC1, COC2, and COC3 (Zheng et al., Cancer Cell 2016). Notably, these molecular subtypes are characterized by a distinct pattern of somatic alterations, activation of unique transcriptional programs, and profound changes in epigenetic patterning. Importantly, COC1-3 status predicts disease course under standard of care therapies—patients with COC1 disease largely have favorable prognosis, those with COC2 disease have intermediate prognosis, and those with COC3 disease have uniformly dismal prognosis.

Tumors with Wnt pathway alterations typically fall in the COC2 and COC3/CIMP-high classes (strategies to identify these classes are detailed in co-pending Pat. Ap. No. PCT/US2020/037039 and WO 2019/108568; each of which is herein incorporated by reference in its entirety).

Wnt pathway activation is associated with upregulation of an SF1-dependent adrenal differentiation transcriptional program. COC2 and COC3/CIMP-high ACC, even without Wnt pathway alterations, possess higher expression of Wnt target genes and adrenal differentiation genes than other ACC (e.g. COC1).

β-catenin coordinates a tissue-specific epigenetic landscape in ACC, that is hyperactive in COC2 and COC3/CIMP-high tumors and required for cell survival. β-catenin coordinates this landscape through a physical interaction with SF1 that activates transcription through binding numerous sites in the genome. Hence, multiple actions of SF1 and β-catenin are interdependent. Experiments described herein demonstrated that it is possible to disrupt the SF1/β-catenin program through two interventions: 1) inhibition of epigenetic modifier EZH2—Inhibition of EZH2 disrupts SF1/β-catenin's interaction with chromatin and erases the SF1/β-catenin-dependent transcriptional program; and 2) Inhibition of epigenetic modifier CREB-binding protein (CBP)—inhibition of CBP induces the same transcriptional changes as inhibition of EZH2, i.e. strong downregulation of SF1/β-catenin target genes.

It was further demonstrated that targeting the SF1/β-catenin program using two agents simultaneously (e.g., inhibitors of EZH2 and CBP) induces synergistic loss of viability, indicating that multiple therapies may be combined at lower doses to limit undesirable and potentially unsafe systemic effects while continuing to target the pathway.

Targeting the tissue-specific β-catenin program is advantageous because it does not affect canonical Wnt signaling and is unlikely to induce system-wide toxicity.

Accordingly, provided herein are methods of treating ACC with Wnt pathway alterations or other evidence of Wnt pathway activation (e.g., COC2 or COC3) with inhibitors that target tissue-specific actions of β-catenin. These include but are not limited to inhibitors of transcription of steroidogenic enzymes or other SF1/β-catenin co-target genes (e.g. including the gene encoding Steroidogenic factor 1 (SF1 itself, NR5A1), inhibitors of epigenetic modifiers that reinforce the SF1/β-catenin program (e.g. inhibitors of EZH2 or CBP), inhibitors of the SF1/β-catenin interaction (e.g. a small molecule that disrupts the SF1/β-catenin binding interface); and combinations of agents that individually disrupt the SF1/β-catenin program.

While the present disclosure is exemplified with ACC, this strategy further finds use with other cancers which bear constitutive activation of β-catenin. β-catenin has been shown to interact with a number of tissue-specific nuclear receptors (e.g. the androgen receptor, estrogen receptor). It is likely that Wnt pathway alterations also augment these tissue-specific programs that are required for cancer development, and may represent a tissue-specific axis of the Wnt pathway.

FIG. 23 exemplifies three classes of therapeutic interventions: A. Interventions that disrupt binding of β-catenin/tissue-specific TF to DNA (e.g., EZH2 inhibitors disrupt binding of SF1/β-catenin complexes to the DNA and disrupt the transcriptional program); B. Interventions that disrupt recruitment of transcriptional machinery or disrupt other mechanisms of transriptional activation (e.g., CBP inhibitor disrupts transcriptional activation driven by SF1/β-catenin); and C. Interventions that disrupt binding between β-catenin/tissue-specific TF (e.g., an inhibitor of the interaction between SF1/β-catenin that leads to downregulation of the SF1/β-catenin-dependent transcriptional program).

The present disclosure is not limited to particular types of inhibitors of the described genes. Examples include but are not limited to, small molecules, nucleic acids, and antibodies. In some embodiments, commercially available inhibitors are utilized.

In some embodiments, the EZH2 small molecule inhibitor is, for example,

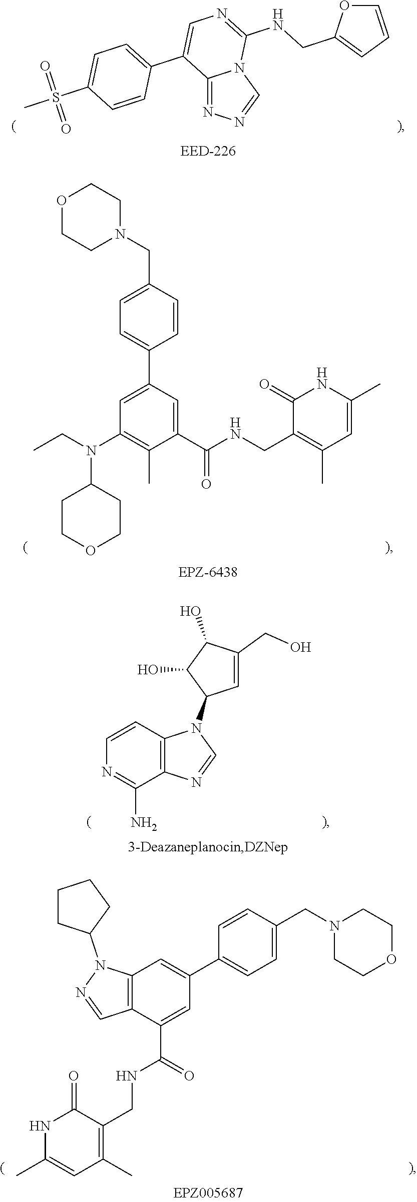

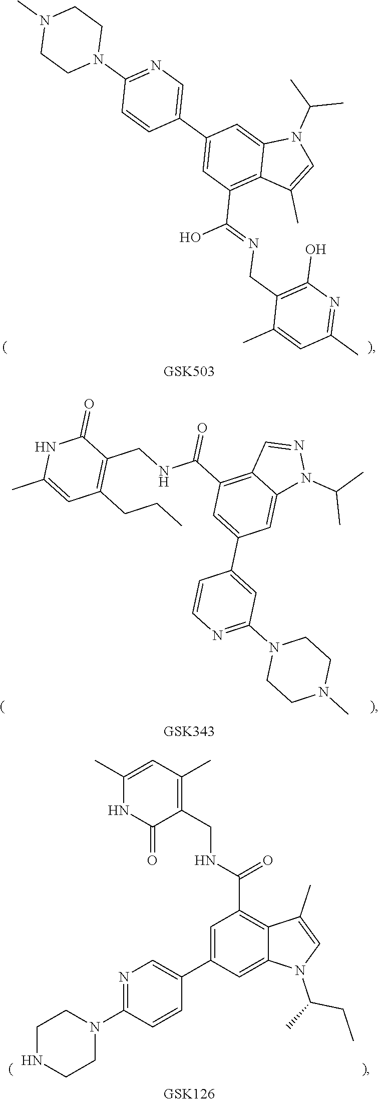

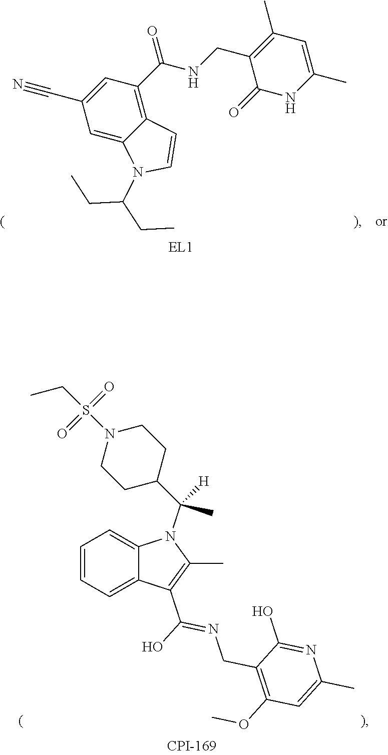

all of which are commercially available (e.g., from ApexBio, Houston, TX).

Small molecule inhibitors of CBP include but are not limited to,

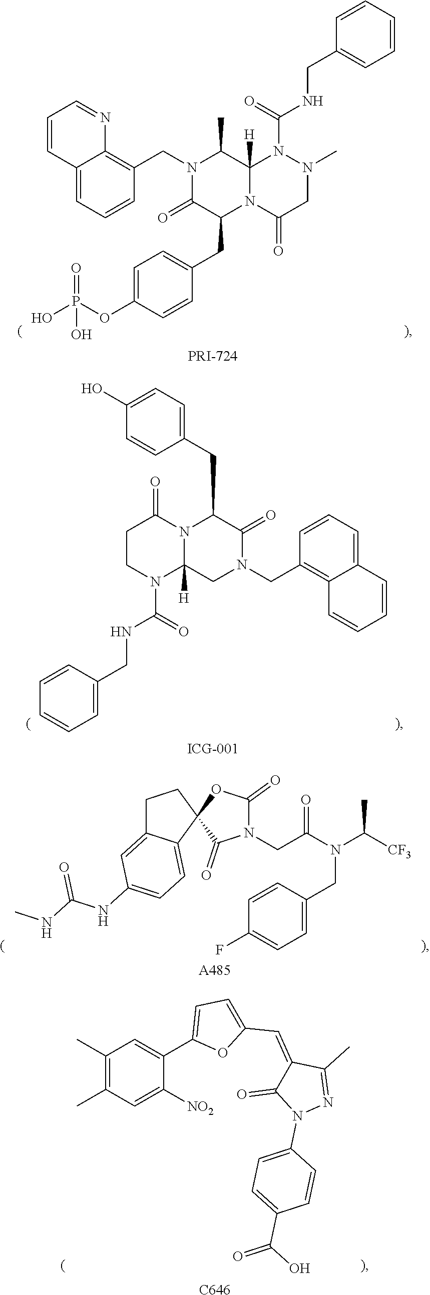

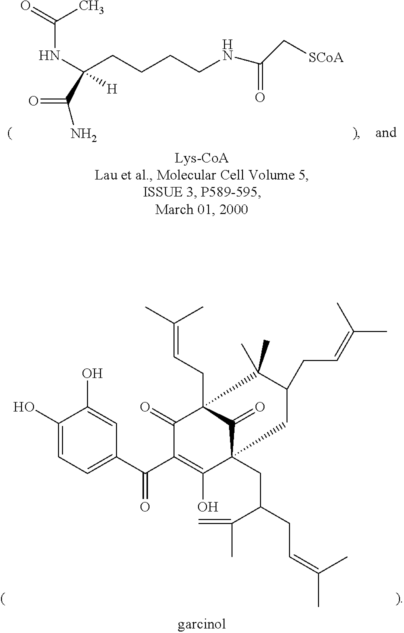

Small molecule inhibitors of SF1 include but are not limited to, SID7969543 and SID7970631 (Madoux et al., Mol Pharmacol. 2008 June; 73 (6): 1776-1784).

In some embodiments, the inhibitor is a nucleic acid. Exemplary nucleic acids suitable for inhibiting expression of the described markers (e.g., by preventing expression of the marker) include, but are not limited to, antisense nucleic acids and RNAi. In some embodiments, nucleic acid therapies are complementary to and hybridize to at least a portion (e.g., at least 5, 8, 10, 11, 12, 13, 14, 15, 16, 17, 18, 19, or 20 nucleotides) of a marker described herein.

In some embodiments, compositions comprising oligomeric antisense compounds, particularly oligonucleotides are used to modulate the function of nucleic acid molecules encoding a marker described herein, ultimately modulating the amount of marker gene expressed. This is accomplished by providing antisense compounds that specifically hybridize with one or more nucleic acids encoding the marker genes. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is decreasing the amount of marker expressed.

In some embodiments, nucleic acids are RNAi nucleic acids. “RNA interference (RNAi)” is the process of sequence-specific, post-transcriptional gene silencing initiated by a small interfering RNA (siRNA), shRNA, or microRNA (miRNA). During RNAi, the RNA induces degradation of target mRNA with consequent sequence-specific inhibition of gene expression.

In “RNA interference,” or “RNAi,” a “small interfering RNA” or “short interfering RNA” or “siRNA” or “short hairpin RNA” or “shRNA” molecule, or “miRNA” an RNAi (e.g., single strand, duplex, or hairpin) of nucleotides is targeted to a nucleic acid sequence of interest, for example, a marker disclosed herein.

An “RNA duplex” refers to the structure formed by the complementary pairing between two regions of an RNA molecule. The RNA using in RNAi is “targeted” to a gene in that the nucleotide sequence of the duplex portion of the RNAi is complementary to a nucleotide sequence of the targeted gene. In certain embodiments, the RNAi is are targeted to the sequence encoding a marker described herein. In some embodiments, the length of the RNAi is less than 30 base pairs. In some embodiments, the RNA can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the RNAi is 19 to 32 base pairs in length. In certain embodiment, the length of the RNAi is 19 or 21 base pairs in length.

In some embodiments, RNAi comprises a hairpin structure (e.g., shRNA). In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

“miRNA” or “miR” means a non-coding RNA between 18 and 25 nucleobases in length which hybridizes to and regulates the expression of a coding RNA. In certain embodiments, a miRNA is the product of cleavage of a pre-miRNA by the enzyme Dicer. Examples of miRNAs are found in the miRNA database known as miRBase.

As used herein, Dicer-substrate RNAs (DsiRNAs) are chemically synthesized asymmetric 25-mer/27-mer duplex RNAs that have increased potency in RNA interference compared to traditional RNAi. Traditional 21-mer RNAi molecules are designed to mimic Dicer products and therefore bypass interaction with the enzyme Dicer. Dicer has been recently shown to be a component of RISC and involved with entry of the RNAi into RISC. Dicer-substrate RNAi molecules are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23:222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional RNAi molecules. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs.

“Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (e.g., about 35 nucleotides upstream and about 40 nucleotides downstream) arise from restriction enzyme sites within the multiple cloning site of the RNAi. As used herein the term “miRNA” encompasses both the naturally occurring miRNA sequences as well as artificially generated miRNA shuttle vectors.

The RNAi can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

The present disclosure further provides pharmaceutical compositions (e.g., comprising the compounds described above). The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

In some embodiments, one or more (e.g., 1, 2, 3, 4, or more) inhibitors of Wnt pathway activation are administered to a subject.

In some embodiments, one or more targeted therapies are administered in combination with an existing therapy for ACC or other cancer. For example, in some embodiments, subjects with COC3 tumors are administered adjuvant cytotoxic chemotherapy (e.g., one or more of etoposide, doxorubicin, cisplatin or other cytotoxic agents). In some embodiments, the COC classification determination is repeated (e.g., during treatment or after surgery).

In some embodiments, agents described herein are screening for activity against ACC (e.g., in vitro drug screening assays or in a clinical study).

### EXPERIMENTAL

The following examples are provided in order to demonstrate and further illustrate certain preferred embodiments and aspects of the present disclosure and are not to be construed as limiting the scope thereof.

### Example 1

### SF1/β-Catenin-Dependent Transcriptional Programming can be Disrupted by Inhibition of EZH2

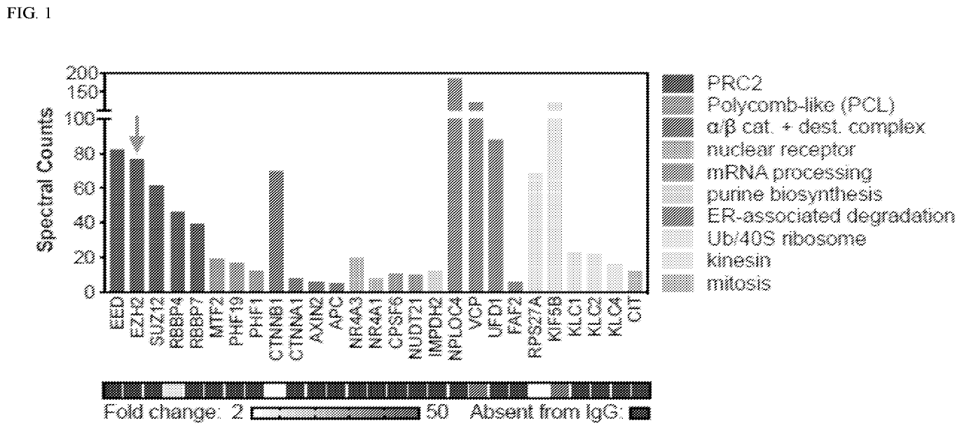

It was observed that EZH2 inhibition (EZH2i) induces demethylation of trimethylated histone H3 lysine 27 (H3K27me3) and dose-dependent loss of viability in the NCI-H295R cell line (data not shown). It was observed that administration of EZH2i at the dose that induces a 50% reduction in viability (IC-50) disrupted a broad spectrum of transcriptional programs in ACC cell line NCI-H295R (6299 genes down, 5460 genes up, adjusted p-value <0.05). This led to an investigation of the consequences of EZH2i on transcriptional programming to determine if it may be partially determined by an off chromatin, non-PRC2 and perhaps tissue-specific role of EZH2. EZH2 was found to have several novel partners, including non-SF1 nuclear receptors known to regulate adrenocortical biology (Bassett et al., 2004), and β-catenin, which possesses the p.S45P mutation and is constitutively active in NCI-H295R (FIG. 1).

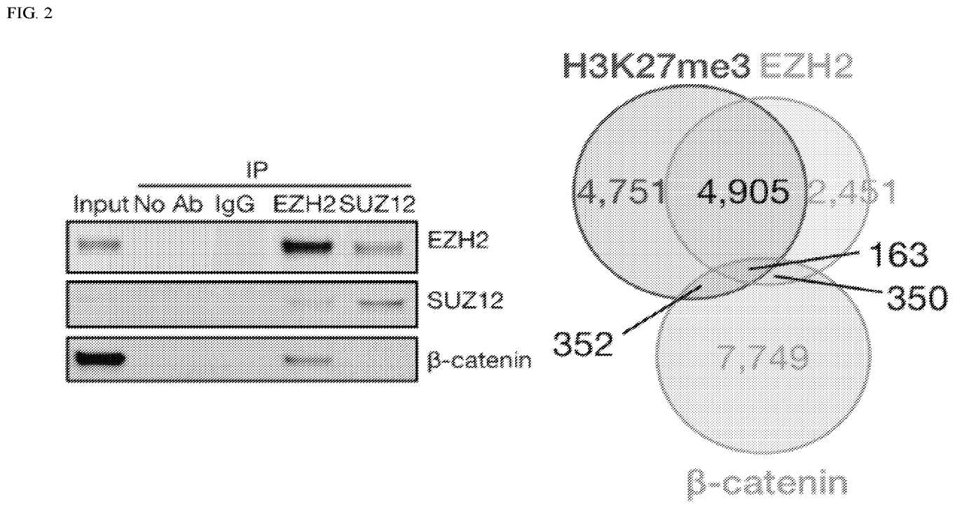

Given its abundance in the EZH2 interactome (FIG. 1) and the well established role of β-catenin in zG to zF lineage conversion and tumorigenesis, studies were focused on the EZH2/β-catenin interaction. Several groups have identified an interaction between EZH2/β-catenin in other tissues, though not in the context of the p.S45P mutation (Anwar et al., 2018; Hoffmeyer et al., 2017; Zhu et al., 2016). In embryonic stem cells, Wnt-dependent induction of mesoderm differentiation may rely on simultaneous β-catenin-dependent activation of Wnt targets genes with β-catenin/PRC2-dependent gene repression (Hoffmeyer et al., 2017). It was therefore investigated if β-catenin is incorporated into the PRC2 directly, or if EZH2/β-catenin are a separate complex. β-catenin's incorporation into the PRC2 was evaluated by performing a PRC2-directed nuclear co-IP. While EZH2 directed nuclear co-IP consistently and successfully retrieved both SUZ12 and β-catenin, SUZ12-directed nuclear co-IP did not retrieve β-catenin. Furthermore, EZH2/H3K27me3/β-catenin possess only minimal overlap on chromatin, indicating EZH2/β-catenin is a nuclear but off chromatin complex (FIG. 2).

Mutations in exon 3 of CTNNB1 (like p. S45P) prevent β-catenin turnover and degradation, and mutant β-catenin therefore accumulates at exceeding high levels in cells expressing the mutation. In the case of NCI-H295R, transcription of CTNNB1 is exclusively from the mutant allele, despite the presence of both wild type and mutant CTNNB1 in the genome. It is therefore possible that β-catenin binding to EZH2 reflects the abundance of this protein in a cancer cell expressing the mutation and may be non-specific or irrelevant for adrenocortical biology. Lack of specificity is unlikely given the criteria for calling protein-protein interactors and that no interaction were observed between β-catenin and SUZ12 or DNMT1 (β-catenin/DNMT1 evaluated by IP-MS). Efforts to affinity purify an EZH2/β-catenin complex out of adrenal tissue were unsuccessful, though one could purify PRC2 by EZH2-directed co-IP on nuclear lysate prepared from 20 flash frozen mouse adrenals.

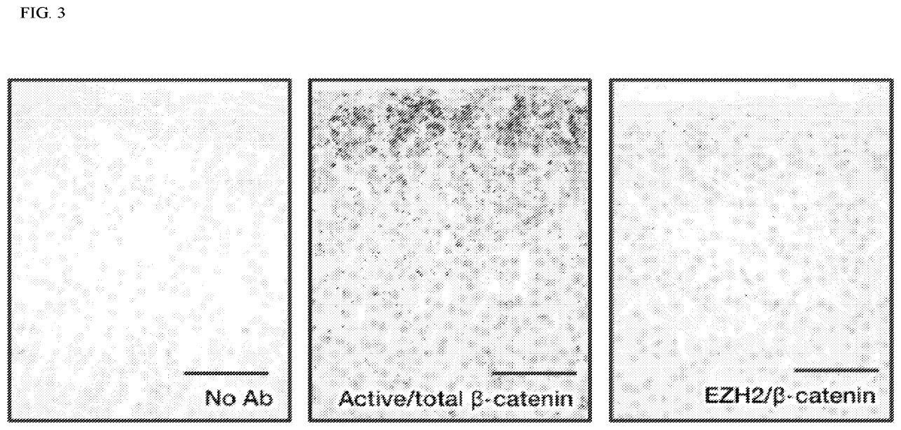

To evaluate a role for this complex in the adrenal cortex in vivo, a technique that enables detection of protein-protein interactions in situ via proximity ligation assay (PLA) was used. PLA allows one to determine zonal and sub-cellular localization of endogenous protein-protein complexes on slides prepared from thin sections of FFPE tissue; each protein-protein interaction (proximity between antibodies detecting proteins can be a maximum of 40 nm apart) is visualized as a dot. PLA on the murine adrenal cortex identified EZH2/β-catenin complexes were nuclear and zonally distributed, with highest abundance in the adrenocortical zG/upper zF and following the Wnt signaling gradient (FIG. 3).

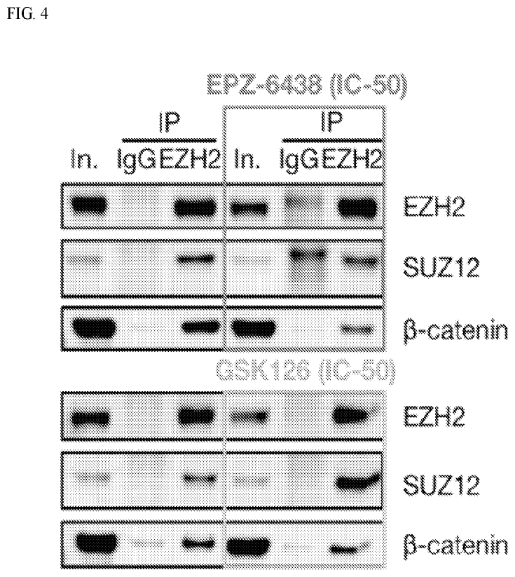

These data indicate that nuclear EZH2/β-catenin may be relevant for adrenocortical biology. Given that EZH2's interactions with its partners are preserved even after EZH2i (FIG. 4), it is possible that this EZH2/β-catenin complex even participates in the cellular response to EZH2 inhibition.

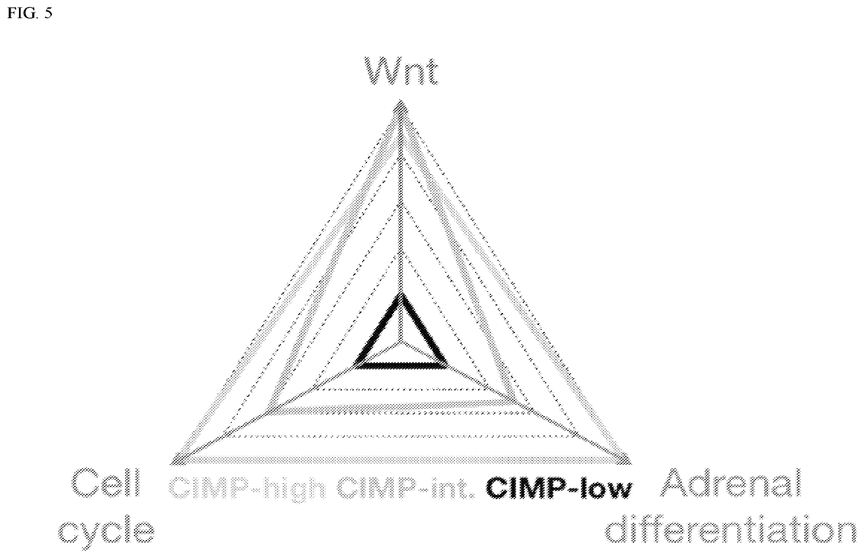

Identification of a zonally distributed EZH2/β-catenin complex and the overlap between programs targeted by EZH2i with physiology led to experiments to determine if EZH2i was disrupting adrenocortical differentiation. In the mouse model of SF1-cre-driven Ezh2 deficiency, mice develop profound defects in zonation, culminating ultimately in a failure of zG to zF lineage conversion, zF hypoplasia, and glucocorticoid insufficiency (Mathieu et al., 2018). Human ACC and ACC cell lines exhibit a spectrum of zF differentiation, Wnt/β-catenin-dependent programming and proliferation, with CIMP-high ACC at the maxima of these three poles (FIG. 5).

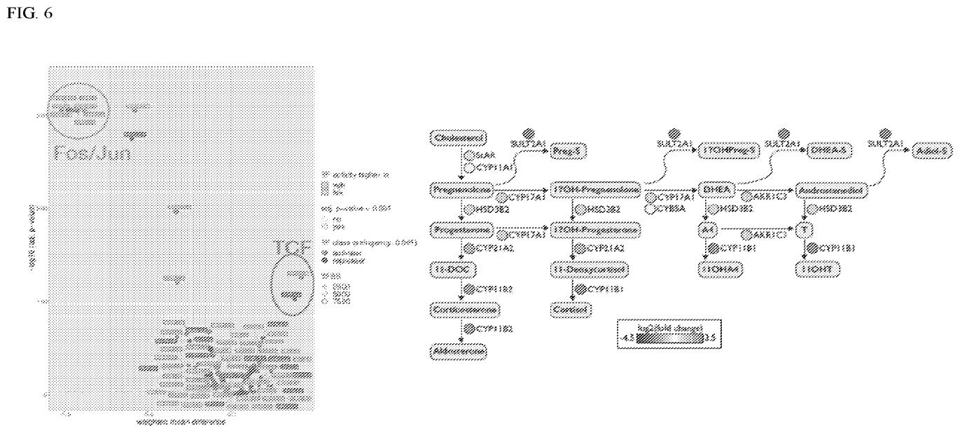

To evaluate if EZH2i disrupted zF differentiation, the NCI-H295R was treated with the zF differentiation agent forskolin and the transcriptome was profiled by RNA-seq. Despite that CIMP-high as a class is at the zF differentiation maximum across human ACC (FIG. 5), the presence of mutant CTNNB1 in this cell line and high levels of nuclear β-catenin are likely to enable faithful responsiveness to forskolin and refractory response to Wnt/β-catenin stimulants, analogously to the Y1 cell line. Indeed, forskolin administration increased expression of zF differentiation genes, shut down expression and chromatin accessibility of zG/canonical Wnt target genes, and potently induced expression of steroidogenic enzymes in NCI-H295R (FIG. 6).

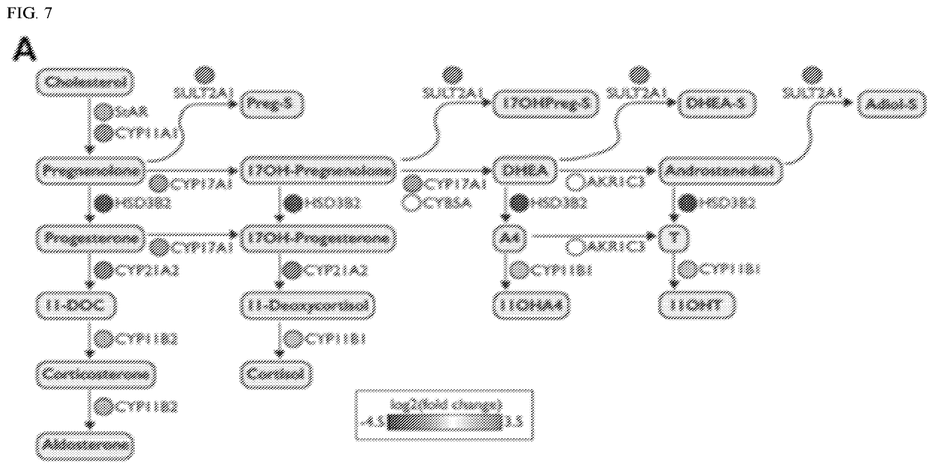

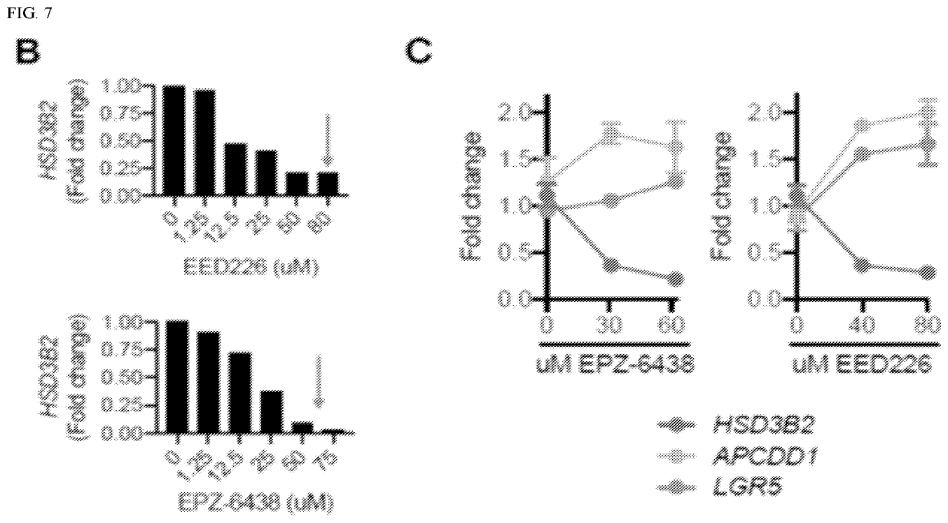

Strikingly, comparison of RNA-seq data from EZH2i and forskolin-treated cells revealed that EZH2i disrupted roughly 70% of genes differentially expressed following forskolin administration, and potently suppressed expression of steroidogenic enzymes. EZH2i-induced suppression of steroidogenic enzymes was dose-dependent and observed with two different classes of EZH2i. Moreover, pretreatment of NCI-H295R with EZH2i prior to forskolin administration diminished forskolin-induced silencing of canonical Wnt targets and induction of steroidogenic enzymes (FIG. 7).

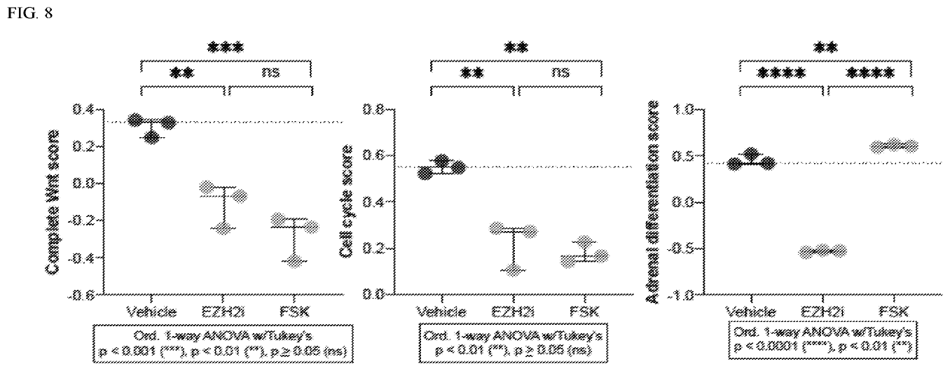

These observations are consistent with a role for EZH2 in programming cellular response to PKA (Mathieu et al., 2018), though likely not through the proposed mechanism of disruption of expression of PKA signaling components given EZH2i induces broad disruption of the transcriptome. Indeed, it was observed that EZH2i reversed all three core transcriptional modules of CIMP-high ACC, while forskolin could only induce differentiation at the expense of proliferation and Wnt-dependent programming (FIG. 8).

In addition to diminishing clonogenic potential and despite failing to modulate the DNA methylome, EZH2i had a potent impact on transcriptional programs that define CIMP-high ACC (FIG. 8). This data indicates that EZH2 coordinates a chromatin landscape that stabilizes the differentiated and Wnt-active CIMPhigh transcriptional state that enables sustained proliferation. Given that part of this transcriptional state reflects Wnt/β-catenin transcriptional activity and that β-catenin is a major binding partner of EZH2 not disrupted by EZH2i, β-catenin's role on chromatin was next investigated.

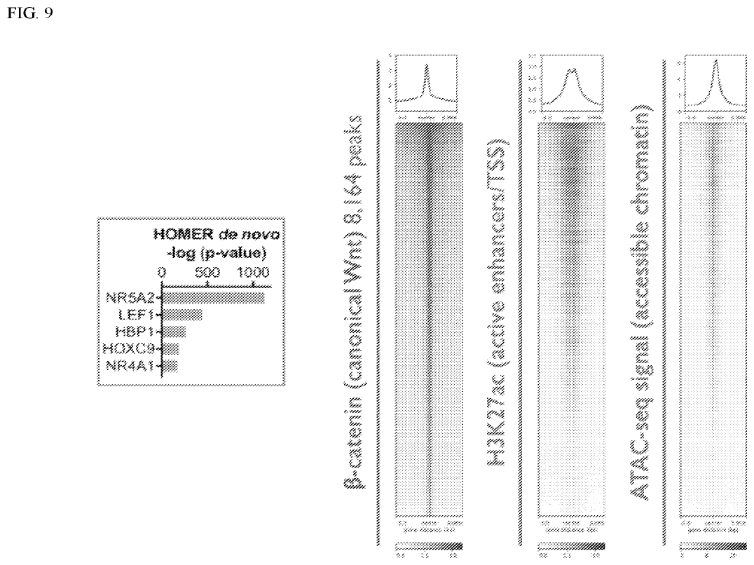

ChIP-seq was performed for β-catenin in NCI-H295R at baseline, and identified That β-catenin principally binds active and accessible chromatin regions. Motif enrichment was performed for β-catenin peaks and a substantial enrichment for the SF1 motif was observed, enriched even more significantly than motifs corresponding to canonical Wnt/β-catenin transcription factors TCF/LEF (FIG. 9).

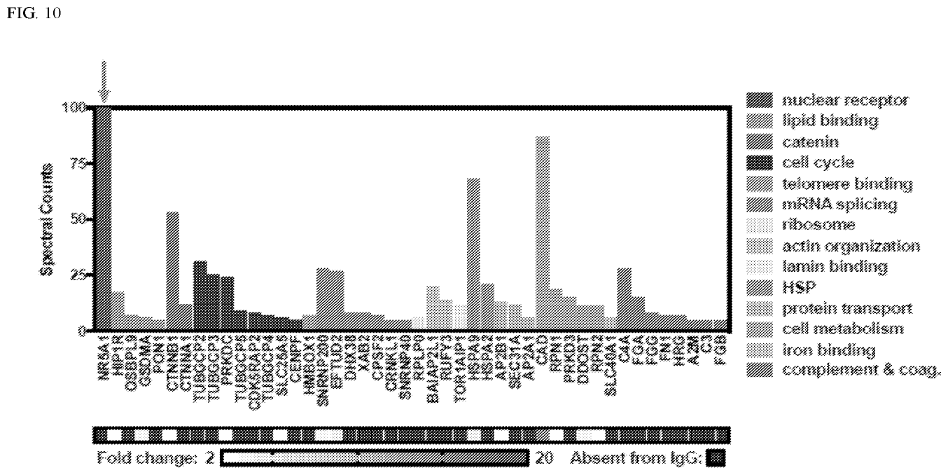

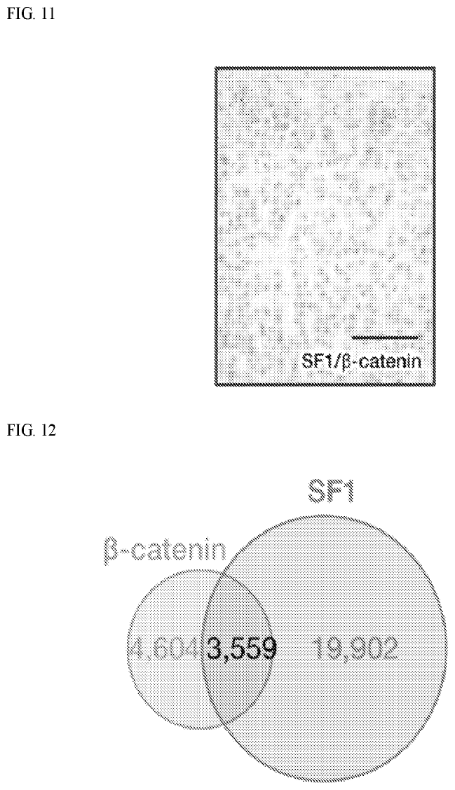

SF1-directed IP-MS revealed that p.S45P β-catenin is also a major binding partner of SF1 in NCI-H295R (FIG. 10). An SF1/β-catenin interaction in the Y1 cell line and adrenal cortex has been previously reported by our group (Gummow et al., 2003), and is thought to regulate gene expression in a context-specific manner (Mizusaki et al., 2003). The presence of zonally distributed SF1/β-catenin complexes in the murine adrenal cortex was identified by PLA (FIG. 11).

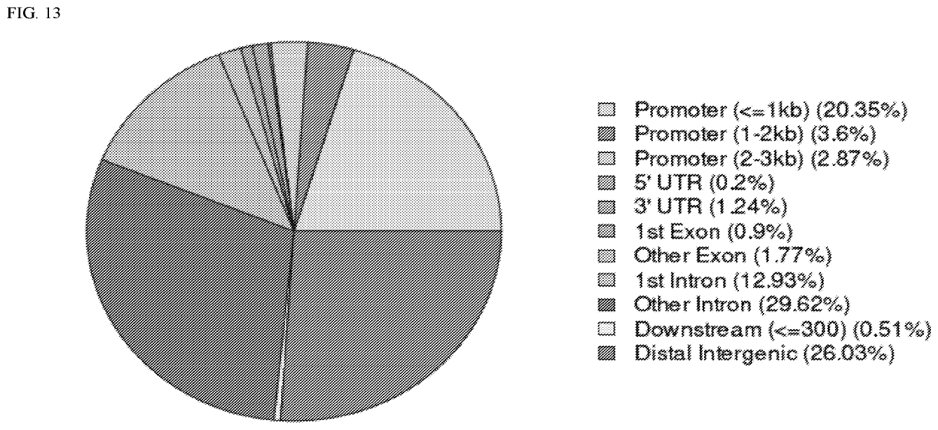

The interaction interface between SF1/β-catenin has been mapped and is believed to reside in the C-terminus of β-catenin, and hence not disrupted by modifications to exon 3 (Mizusaki et al., 2003). An interaction between SF1 and p.S45P β-catenin has not previously been described, nor is it well understood if SF1/β-catenin effect global coordination of gene expression programs or simply co-occupy few loci. ChIP-seq was performed for SF1, and it was identified that SF1 binds accessible and active chromatin regions. There was also substantial overlap between SF1 and β-catenin binding sites (FIG. 12), and SF1/β-catenin sites encompassed predominantly distal CREs (FIG. 13). Given the strong overlap of SF1 and β-catenin with H3K27ac and accessible chromatin, these data indicate that SF1/β-catenin coordinate transcriptional programming genome-wide, predominantly through enhancer programming.

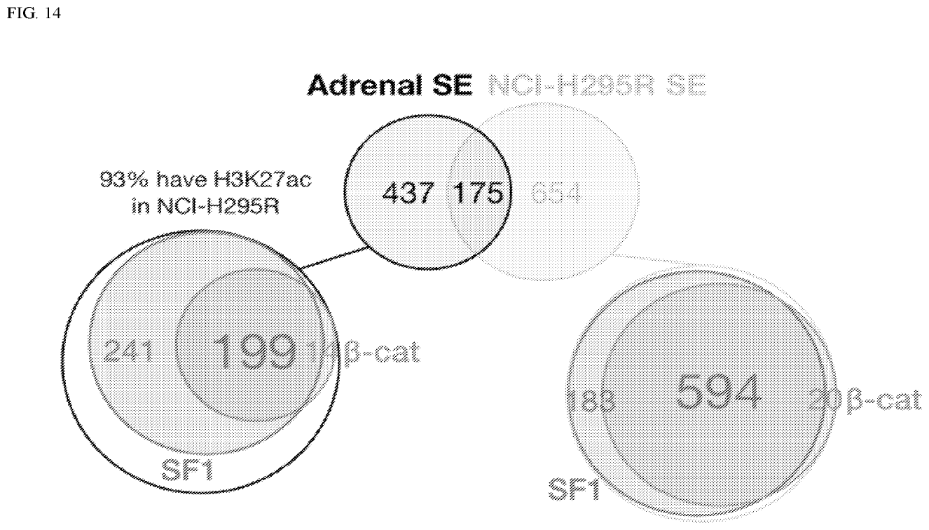

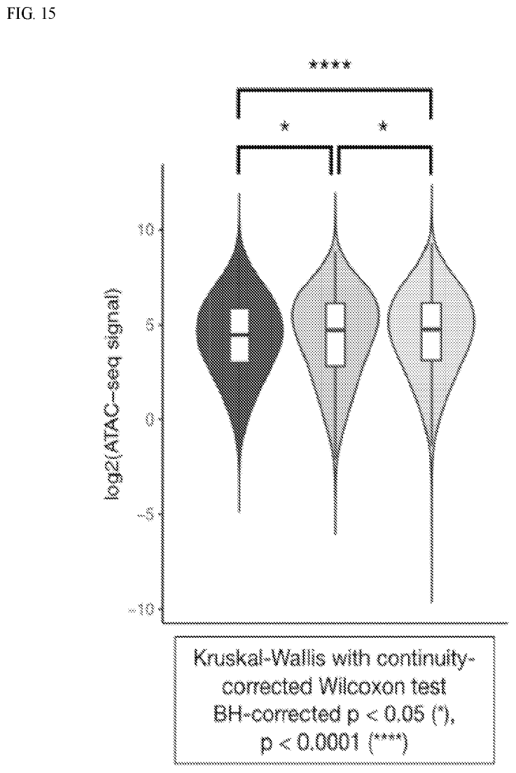

Enhancers serve as critical nodes for regulation of gene expression, as a single enhancer may coordinate the expression of many promoters, and therefore many genes. A special class of enhancers, super-enhancers (SE), with high density occupancy of Mediator (a complex that bridges enhancer/promoter contacts, (Kagey et al., 2010)) has been implicated in lineage-specific programming (Hnisz et al., 2013; Sabari et al., 2018; Whyte et al., 2013; Zamudio et al., 2019). SEs also possess high density deposition of H3K27ac and occupancy of lineage-defining transcription factors, and drive pervasive cell-of-origin transcriptional programs in development and disease (Hnisz et al., 2013; Hnisz et al., 2015; Whyte et al., 2013). Bioinformatically, SEs can be identified by “stitching” nearby enhancer and ranking them by H3K27ac density (Lovén et al., 2013; Pott and Lieb, 2015; Whyte et al., 2013). It was determined if SE programming in CIMP-high ACC is coordinated by SF1/β-catenin, and if this coordination reflects physiological programming or is cancer specific. SE analysis was performed on NCI-H295R ChIP-seq, and NCI-H295R SE was compared to physiological adrenal SE. >90% of adrenal SE retain H3K27ac deposition in NCI-H295R, but ˜70% of these enhancers are demoted from SE status in this cell line. ˜80% of SE in NCI-H295R are novel, and ˜70% of all NCI-H295R SE are bound by both SF1 and β-catenin. This data indicated that SF1/β-catenin together are coordinating a master switch of adrenal differentiation in NCIH295R, and represents a major departure from SE regulation in the physiological adrenal gland, wherein only ˜30% of SE possess SF1/β-catenin occupancy in NCI-H295R (FIG. 14).

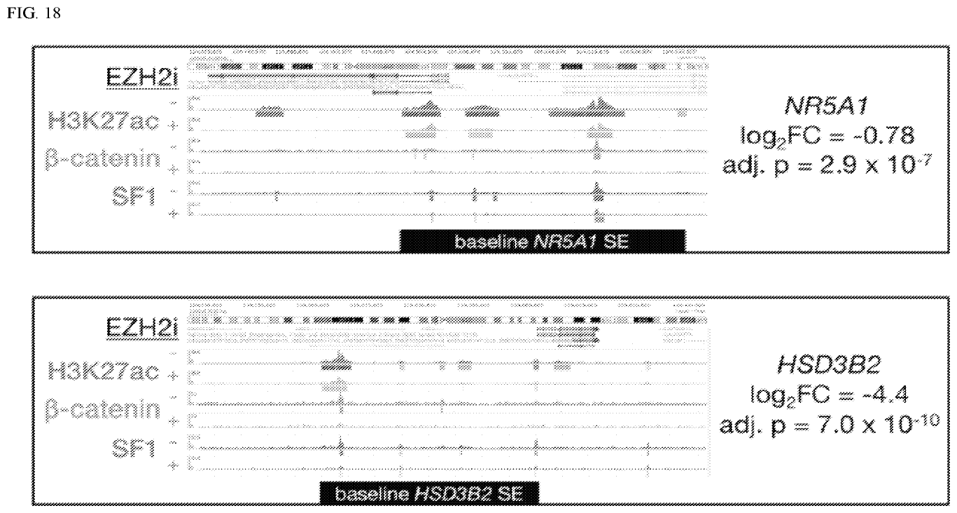

As expected considering the predicted tissue-defining roles of SEs (Hnisz et al., 2013; Sabari et al., 2018; Whyte et al., 2013; Zamudio et al., 2019), SF1/β-catenin SEs regulate expression of many genes that are critical for adrenocortical and steroidogenic identity, including HSD3B2 and NR5A1 itself (FIG. 18). These SEs are also present in the physiological adrenal gland.

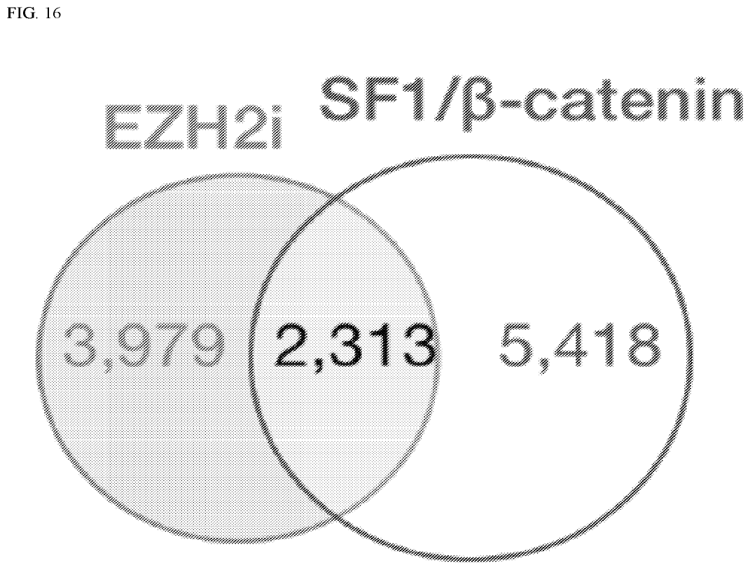

Finally, to extend this analysis to ACC more broadly, it was determined if SF1/β-catenin sites are differentially accessible across CIMP classes in ACC-TCGA, as would be expected given the augmentation of both Wnt/β-catenin and adrenocortical differentiation in these tumors (FIG. 5). Though there are only few ACC-TCGA samples with ATAC-seq profiling and our analyses are indeed preliminary, we observed a significant increase in accessibility of SF1/β-catenin co-targets in CIMP-high tumors (FIG. 16).

Taken together, these studies in NCI-H295R identify a novel SF1/β-catenin dependent differentiation axis that exists in physiological tissue and is augmented in CIMP-high ACC. Identifying SF1/β-catenin-dependent SE in physiological adrenal as well as zonally distributed SF1/β-catenin and EZH2/β-catenin indicates that these complexes may be present prior to or in early stages of carcinogenesis and are selected for through dysplasia and malignancy. It was therefore next investigated if SF1/β-catenin and EZH2/β-catenin accompany murine adrenocortical carcinogenesis and identified these complexes are present in early and late stages of tumorigenesis.

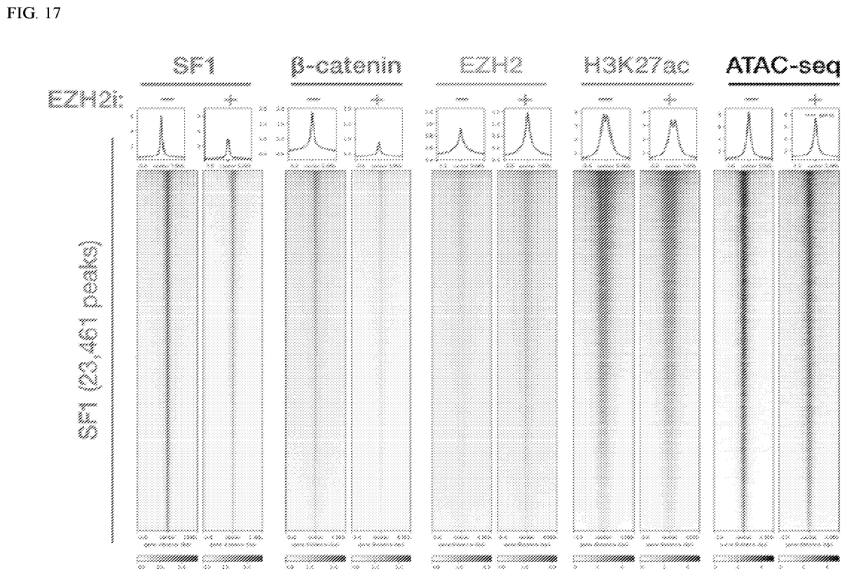

The presence of EZH2/β-catenin and SF1/β-catenin in vivo was compelling, given the observation that EZH2/β-catenin is an off-chromatin complex that persists with EZH2i. EZH2i reversed adrenal differentiation in CIMP-high ACC, which is likely nearly entirely coordinated by SF1/β-catenin. Furthermore, many genes that are regulated by SF1/β-catenin enhancers are repressed by EZH2i (FIG. 16). These data led to a hypothesis that EZH2i may disrupt SF1/β-catenin recruitment genome-wide, which was evaluated by ChIP-seq. IT was observed that SF1 and β-catenin were globally evicted at SF1 targets by EZH2i, at the expense of aberrant EZH2 recruitment (FIG. 17).

Given the preservation of EZH2/β-catenin following EZH2i (FIG. 5) and the impact of EZH2i on EZH2 recruitment genome-wide (FIG. 17), it was speculated that EZH2i expunges β-catenin from chromatin secondary to the “excess” of EZH2 induced by its eviction from H3K27me3 domains. Given that a direct interaction between EZH2 and SF1 (FIGS. 1, 10) was not observed, it was difficult to determine why EZH2i also disrupted SF1 solo programming. However, inspecting the prototype HSD3B2 and NR5A1 SEs before and after EZH2i, it was observed that EZH2i disrupted SF1 and β-catenin binding to these loci and this was associated with a decrease in gene expression (FIG. 18).

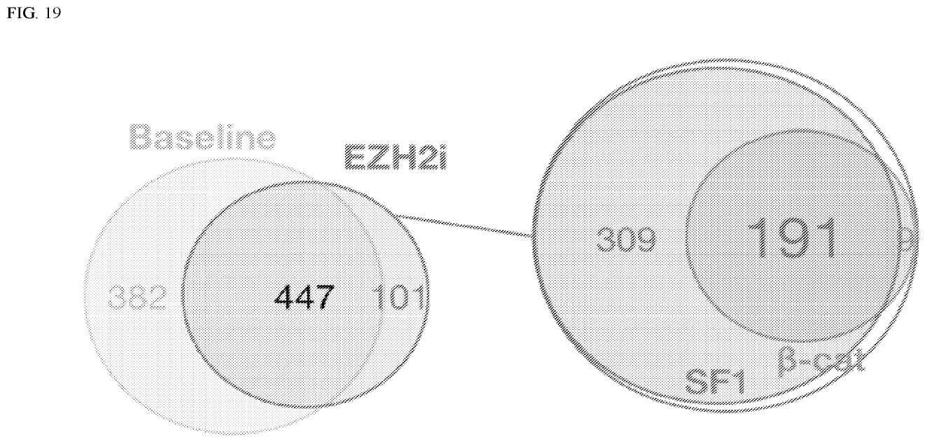

This indicated that the impact of EZH2i on SF1 recruitment genome-wide may be a consequence of disrupted SE programming. Examining super enhancers more broadly, we observed that EZH2i downgrades nearly half of all SE, and residual SE lose SF1/β-catenin coordinate control (FIG. 19).

These data converge on the idea that, through manipulation of the SF1/β-catenin axis, the most direct and immediate consequence of EZH2i is on disruption of SE programming.

### Example 2

### SF1/β-Catenin-Dependent Transcriptional Programming can be Disrupted by Inhibition of CBP+/−EZH2

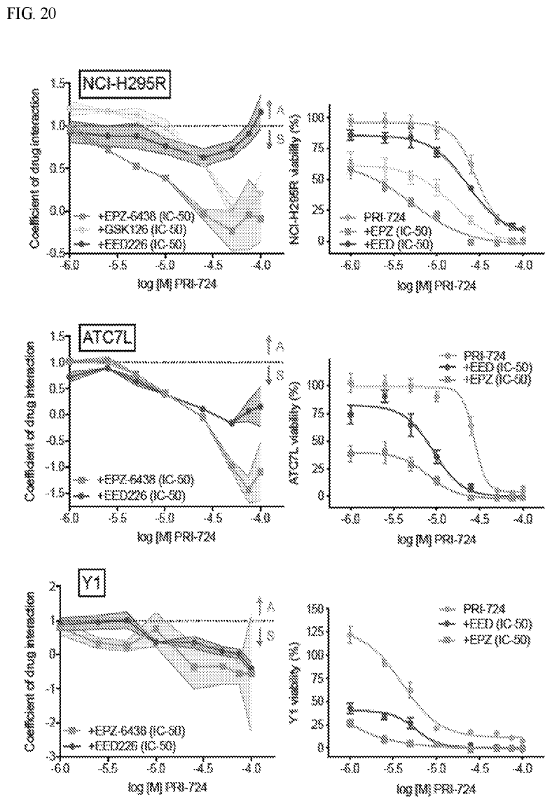

ACC cell lines were treated with a specific and irreversible inhibitor of the H3K27 acetyltransferase CBP (PRI-724 (Kahn, 2014)). CBP regulates H3K27ac deposition genome-wide including at enhancers, and CBP-dependent H3K27ac deposition is required for enhancer activity (Merika et al., 1998; Raisner et al., 2018). Cell lines received the CBP inhibitor (CBPi) either alone or combination with EZH2i at the determined IC-50 dose for that cell line. It was observed that combination EZH2i/CBPi was synergistic in all ACC cell lines tested, indicating that these drugs target the same biological program (FIG. 20).

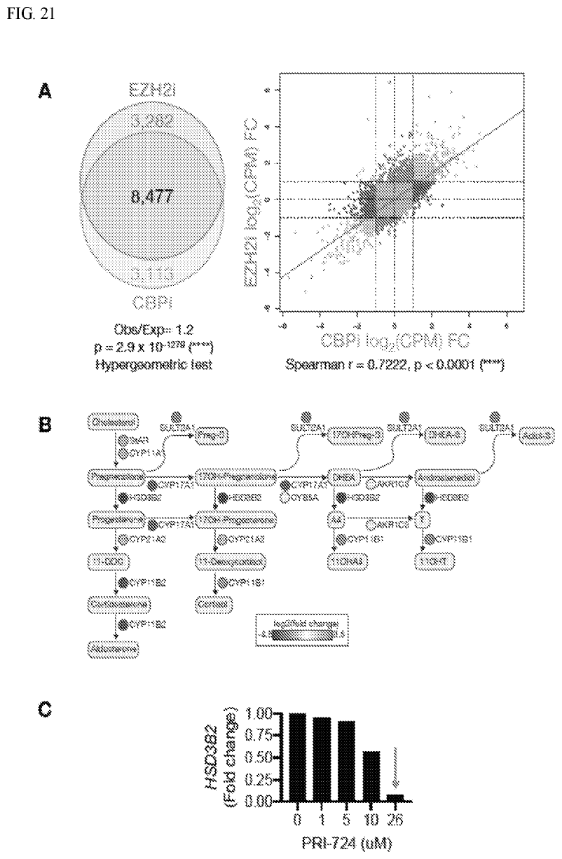

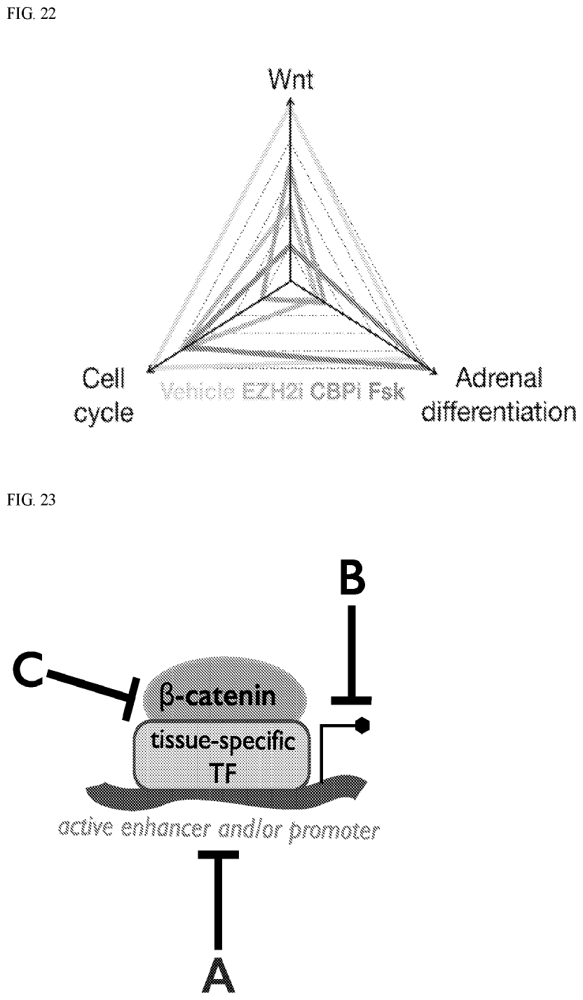

The NCI-H295R response to CBPi was evaluated by RNA-seq and compared to the NCI-H295R response to EZH2i. Redundant and highly correlated effects of CBPi and EZH2i on the NCI-H295R transcriptome were observed, including a potent and dose-dependent downregulation of steroidogenic enzymes (FIG. 21). Similarly to EZH2i, CBPi induces downregulation of all core modules that define CIMP-high ACC (FIG. 22). Taken together, the results point to adrenocortical differentiation as a targetable therapeutic vulnerability selected for in CIMP-high ACC.

### Example 3

### Tissue-Specific β-Catenin Complexes Prevail Across Human and Murine ACC and can be Targeted by EZH2 Inhibition to Decrease ACC Growth

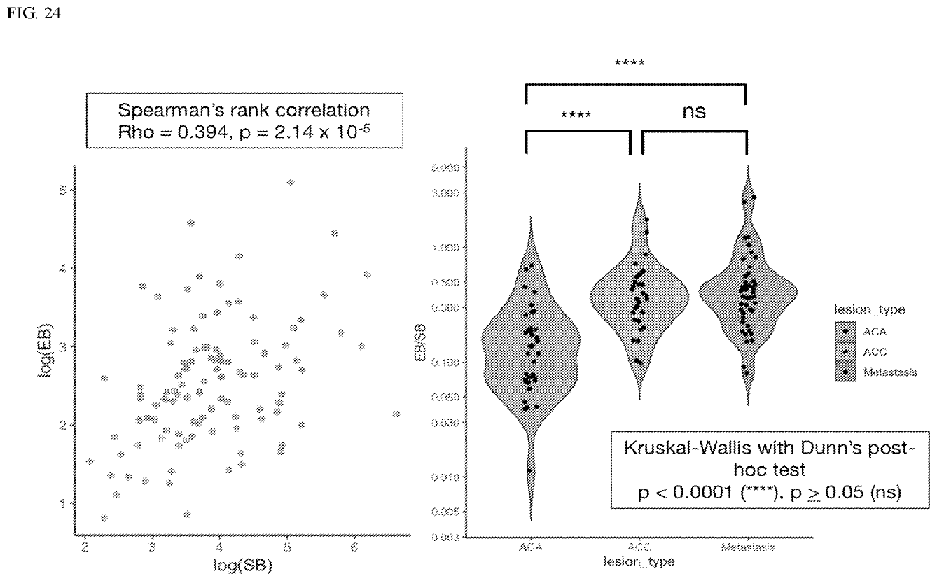

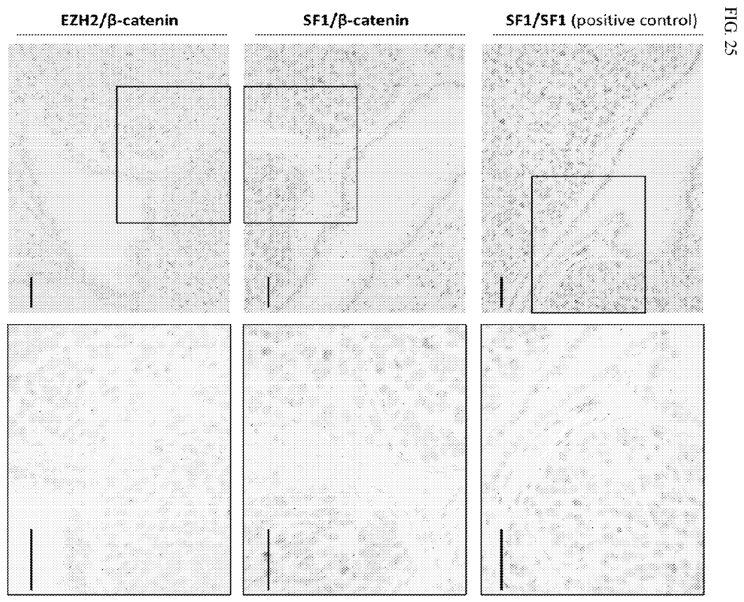

Proximity ligation assay (PLA) technology was applied to the human physiologic adrenal cortex, and a tissue microarray (TMA) of benign adrenocortical tumors and primary and metastatic ACC to measure tissue-specific β-catenin complexes. In the human adrenal, a zonal distribution of nuclear SF1/β-catenin complexes also mirroring the Wnt/β-catenin gradient, and infrequent nuclear EZH2/β-catenin complexes mirroring the rarity of EZH2 expression in the human adrenal cortex was observed. In the TMA retention of β-catenin-containing complexes through metastatic disease, with increased abundance of EZH2/β-catenin complexes relative to SF1/β-catenin complexes in malignancy was observed (FIG. 24). In a genetically engineered mouse model of adrenal cancer, in which adrenocortical cells express a constitutively active β-catenin and deletion in a gene encoding a negative regulator of the cell cycle (p53) (Borges et al. Oncogene 2020), abundance of EZH2/β-catenin and SF1/β-catenin complexes was tracked. Hyperplastic transformation and metastatic seeding of cells uniformly expressing EZH2/β-catenin and SF1/β-catenin complexes was observed (FIG. 25). These data demonstrate that persistence of EZH2/β-catenin and SF1/β-catenin complexes is conserved across murine and human adrenocortical carcinogenesis, indicating that the programs correlated with or directly coordinated by these complexes are subject to positive selection through all phases of β-catenin-dependent adrenal cancer evolution.

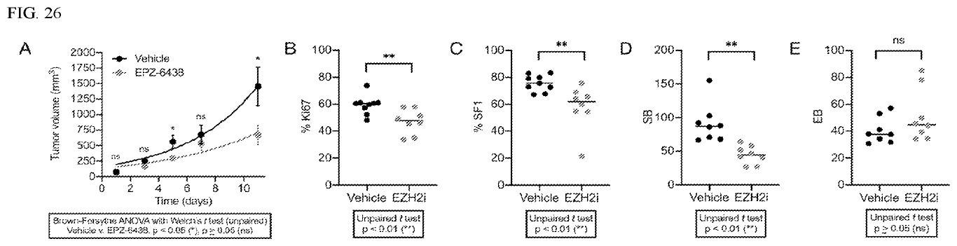

To test if disruption of SF1/β-catenin complexes in vivo can cause dedifferentiation (Example 1 and 2) and disrupt tumor growth, the impact of EZH2 inhibition on a xenograft model derived from the Borges et al. Oncogene 2020 genetically engineered mouse model detailed in FIG. 25 was assayed. EZH2 inhibition decreased tumor growth and proliferation rate, decreased abundance of SF1/β-catenin complexes without affecting EZH2/β-catenin complexes, and decreased SF1 expression (FIG. 26). These data are consistent with the cellular consequences of EZH2 inhibition characterized in vitro (Examples 1 and 2).

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in the medical sciences are intended to be within the scope of the following claims.

1. A method for treating adrenocortical carcinoma (ACC), comprising:
administering at least one agent that blocks activation of the Wnt/β-catenin pathway to a subject identified as having COC2 or COC3/CIMP-high ACC.

2. The method of claim 1, wherein said ACC exhibits constitutive activation of the Wnt/β-catenin pathway.

3. A method for treating cancer, comprising:
administering at least one agent that blocks activation of the Wnt/β-catenin pathway to a subject identified as having a cancer that exhibits constitutive activation of the Wnt/β-catenin pathway.

4. The method of any of the preceding claims, wherein said agent disrupts binding of SF1/β-catenin complexes to DNA; disrupts transcriptional activation of SF1/β-catenin, and/or disrupts binding of SF1 to β-catenin.

5. The method of any of the preceding claims, wherein said agent is an EZH2 inhibitor.

6. The method of any of the preceding claims, wherein said agent is an CBP inhibitor.

7. The method of any of the preceding claims, wherein said method comprising administering an EZH2 inhibitor and a CBP inhibitor.

8. The method of any of the preceding claims, wherein said agent is an SF1 inhibitor.

9. The method of any of the preceding claims, wherein said agent is selected from the group consisting of an antibody, a nucleic acid, and a small molecule.

10. The method of claim 9, wherein said EZH2 inhibitor is selected from the group consisting of EED226, EPZ-6438, 3-Deazaneplanocin, DZNep, EPZ005687, GSK503, GSK343, GSK126, EL1, and CPI-169.

11. The method of claim 9, wherein said CBP inhibitor is selected from the group consisting of ICG-001, PRI-724, A485, C646, 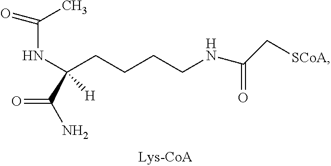
and garcinol.

12. The method of claim 9, wherein said SF1 inhibitor is selected from the group consisting of SID7969543 and SID7970631.

13. The use of an agent that blocks activation of the Wnt/β-catenin pathway to treat ACC in a subject identified as having COC2 or COC3/CIMP-high ACC.

14. An agent that blocks activation of the Wnt/β-catenin pathway for use in treating ACC in a subject identified as having a COC2 or COC3/CIMP-high ACC.

15. A method for treating cancer, comprising:
administering an agent that blocks activation of the Wnt/β-catenin pathway to a subject identified as having constitutive activation of the Wnt/β-catenin pathway in a sample isolated from said subject.

16. The method of claim 15, wherein said cancer is COC2 or COC3/CIMP high ACC.

17. A method for treating cancer in a subject, comprising:
a) identifying the subject constitutive activation of the Wnt/β-catenin pathway by obtaining or having obtained a sample from the subject; and measuring the level of activation of the Wnt/β-catenin pathway in the sample; and
b) administering an agent that blocks activation of the Wnt/β-catenin pathway to said subject when said sample exhibits constitutive activation of the Wnt/β-catenin pathway.

18. A method for treating cancer in a subject, comprising
a) determining the level of activation of the Wnt/β-catenin pathway in a sample from the subject;
b) identifying subjects with constitutive activation of the Wnt/β-catenin pathway in the sample; and
c) administering an agent that blocks activation of the Wnt/β-catenin pathway to said subject identified as having constitutive activation of the Wnt/β-catenin pathway.

19. The method of claim 17 or 18, wherein said sample is a sample of cancer cells or tissue.

20. The method of claim 17 or 18, wherein said cancer is COC2 or COC3/CIMP high ACC.
